(19) United States
(12) Patent Application Publication
(10) Pub. No.: US 2025/0169790 A1
(43) Pub. Date: May 29, 2025

(54) ULTRASOUND DIAGNOSTIC APPARATUS AND PROGRAM

(71) Applicant: FUJIFILM Corporation, Tokyo (JP)

(72) Inventors: Rei YOKOSAWA, Tokyo (JP); Teiichiro IKEDA, Tokyo (JP)

(73) Assignee: FUJIFILM Corporation, Tokyo (JP)

(21) Appl. No.: 18/959,457

(22) Filed: Nov. 25, 2024

(30) Foreign Application Priority Data
Nov. 27, 2023 (JP) 2023-200194

(51) Int. Cl. A61B 8/08

(52) U.S. Cl. CPC A61B 8/08; A61B 8/5223

(57) ABSTRACT

An acquisition unit (for example, a transmission/reception unit and an image generation unit) acquires a first ultrasound image of a subject according to a first imaging condition. A recognition unit executes processing of recognizing a cross section on the first ultrasound image, and outputs a plurality of candidates for the cross section and a reliability degree of the recognition for each of the candidates. In a case where a state where the reliability degree of the plurality of candidates having higher reliability degrees satisfies a threshold value condition is continued for a predetermined time, the acquisition unit acquires a second ultrasound image of the subject according to a second imaging condition according to the recognized cross section, the second imaging condition being different from the first imaging condition.

### CROSS-REFERENCE TO RELATED APPLICATIONS

This application claims benefit of priority to Japanese Patent Application No. 2023-200194 filed Nov. 27, 2023, the entire contents of which are incorporated herein by reference.

### BACKGROUND OF THE INVENTION

### 1. Field of the Invention

The present disclosure relates to an ultrasound diagnostic apparatus and a program.

### 2. Description of the Related Art

There is known a technique of recognizing a cross section obtained by imaging based on an ultrasound image acquired by transmission and reception of ultrasound waves and executing image processing according to the recognized cross section.

JP2022-551143A discloses a system that performs imaging setting optimized for a specific view.

### SUMMARY OF THE INVENTION

On the other hand, in processing of recognizing a cross section, a plurality of candidates for the cross section may be estimated. In this case, it is not always possible to estimate a candidate having a high reliability degree. In this case, a user such as a doctor or a medical technician needs to perform work such as adjustment of imaging conditions for ultrasonography such that an ultrasound image suitable for an examination is acquired. As a result, a burden on the user increases. For example, in a case where the adjustment is performed for each of a plurality of cross sections, work of the user is increased by the adjustment, and as a result, a burden on the user is increased.

An object of the present disclosure is to reduce a burden on a user to change imaging conditions in order to acquire an ultrasound image suitable for an examination.

According to an aspect of the present disclosure, there is provided an ultrasound diagnostic apparatus comprising: an acquisition unit that acquires a first ultrasound image of a subject according to a first imaging condition; and a recognition unit that executes processing of recognizing a cross section on the first ultrasound image and outputs a plurality of candidates for the cross section and a reliability degree of the recognition for each of the candidates, in which, in a case where a state where the reliability degree of the plurality of candidates having higher reliability degrees satisfies a threshold value condition is continued for a predetermined time, the acquisition unit acquires a second ultrasound image of the subject according to a second imaging condition according to the recognized cross section, the second imaging condition being different from the first imaging condition.

The reliability degree of the plurality of candidates having higher reliability degrees may be a sum of the reliability degrees calculated by adding the reliability degree of each candidate in an order from a candidate having a highest reliability degree. The case where the state where the reliability degree of the plurality of candidates having higher reliability degrees satisfies the threshold value condition is continued for the predetermined time may be a case where a state where the sum is within a threshold value range is continued for a predetermined time.

The case where the state where the reliability degree of the plurality of candidates having higher reliability degrees satisfies the threshold value condition is continued for the predetermined time may be a case where a state where ranks of the plurality of candidates having higher reliability degrees are not changed is continued for a predetermined time and the reliability degree of each of the plurality of candidates having higher reliability degrees is lower than a threshold value.

The second imaging condition may be an imaging condition corresponding to a candidate of which the reliability degree is higher than a threshold value among the plurality of candidates having higher reliability degrees.

The second imaging condition may be an imaging condition corresponding to a candidate having a highest reliability degree among the plurality of candidates having higher reliability degrees.

The recognition unit may further execute processing of recognizing a cross section on the second ultrasound image, and calculate a reliability degree of the recognition, and the acquisition unit may further acquire the second ultrasound image according to the second imaging condition in a case where the reliability degree calculated from the second ultrasound image is higher than the reliability degree calculated from the first ultrasound image.

The acquisition unit may further continue ultrasonography according to the first imaging condition in a case where the reliability degree calculated from the second ultrasound image is equal to or lower than the reliability degree calculated from the first ultrasound image.

The ultrasound diagnostic apparatus may further comprise: a controller that displays the first ultrasound image and the second ultrasound image on a display. The acquisition unit may acquire an ultrasound image according to an imaging condition selected by a user from the first imaging condition and the second imaging condition.

The second imaging condition may be a condition related to image processing, and the acquisition unit may acquire the second ultrasound image by executing image processing according to the second imaging condition, on data acquired according to the first imaging condition.

According to another aspect of the present disclosure, there is provided a program causing a computer to function as: an acquisition unit that acquires a first ultrasound image of a subject according to a first imaging condition; and a recognition unit that executes processing of recognizing a cross section on the first ultrasound image and outputs a plurality of candidates for the cross section and a reliability degree of the recognition for each of the candidates, in which, in a case where a state where the reliability degree of the plurality of candidates having higher reliability degrees satisfies a threshold value condition is continued for a predetermined time, the acquisition unit acquires a second ultrasound image of the subject according to a second imaging condition according to the recognized cross section, the second imaging condition being different from the first imaging condition.

According to the present disclosure, it is possible to reduce a burden on a user to change imaging conditions in order to acquire an ultrasound image suitable for an examination.

### BRIEF DESCRIPTION OF THE DRAWINGS

FIG. 1 is a block diagram illustrating an example of a configuration of an ultrasound diagnostic apparatus according to an embodiment.

FIG. 2 is a graph showing a temporal change of a reliability degree of recognition of a cross section.

FIG. 3 is a graph showing a temporal change of a reliability degree of recognition of a cross section.

FIG. 4 is a graph showing a temporal change of a sum of the reliability degrees.

FIG. 5 is a graph showing a temporal change of a moving average value of the sum of the reliability degrees.

FIG. 6 is a graph showing a temporal change of a reliability degree of recognition of a cross section.

FIG. 7 is a graph showing a temporal change of a reliability degree of recognition of a cross section.

FIG. 8 is a graph showing a temporal change of a reliability degree of recognition of a cross section.

FIG. 9 is a graph showing a temporal change of a reliability degree of recognition of a cross section.

### DESCRIPTION OF THE PREFERRED EMBODIMENTS

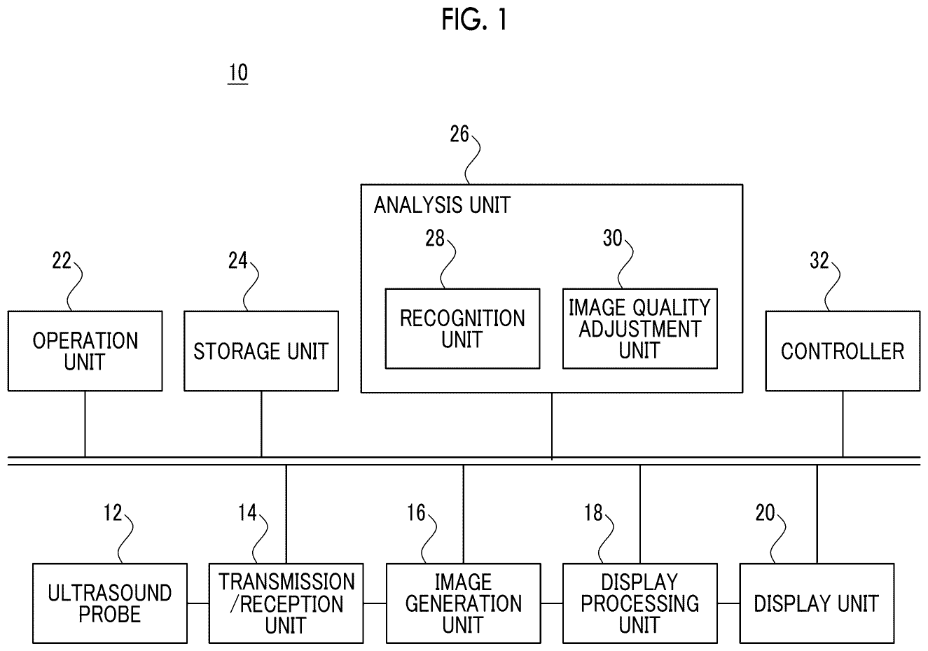

An ultrasound diagnostic apparatus 10 according to an embodiment will be described with reference to FIG. 1. FIG. 1 is a block diagram illustrating an example of a configuration of the ultrasound diagnostic apparatus 10.

The ultrasound diagnostic apparatus 10 generates ultrasound image data by transmitting and receiving ultrasound waves using an ultrasound probe 12. For example, the ultrasound diagnostic apparatus 10 generates ultrasound image data representing tissues inside a subject by transmitting ultrasound waves into a subject and receiving the ultrasound waves reflected from the inside of the subject.

The ultrasound probe 12 is a device that transmits and receives ultrasound waves. The ultrasound probe 12 includes, for example, a 1D array oscillator. The 1D array oscillator includes a plurality of ultrasound oscillators arranged in one dimension. Ultrasound beams are formed by the 1D array oscillator, and electron scanning with the ultrasound beams is repeatedly performed. Thereby, a scanning cross section is formed in a living body for each electron scanning. The scanning cross section corresponds to a two-dimensional echo data acquisition space. The ultrasound probe 12 may include a 2D array oscillator formed by two-dimensionally arranging a plurality of ultrasound oscillators. In a case where ultrasound beams are formed by the 2D array oscillator and the electron scanning with the ultrasound beams is repeatedly performed, the scanning cross section as the two-dimensional echo data acquisition space is formed for each electron scanning. In a case where the scanning with the ultrasound beams is two-dimensionally performed, a three-dimensional space as a three-dimensional echo data acquisition space is formed. As a scanning method, sector scanning, linear scanning, convex scanning, or the like is used.

A transmission/reception unit 14 functions as a transmission beam former and a reception beam former. In the transmission, the transmission/reception unit 14 supplies a plurality of transmission signals having a certain delay relationship to the plurality of ultrasound oscillators included in the ultrasound probe 12. Thereby, transmission beams of the ultrasound waves are formed. In the reception, reflected waves (that is, RF signals) from the living body are received by the ultrasound probe 12. Thereby, a plurality of reception signals are output from the ultrasound probe 12 to the transmission/reception unit 14. The transmission/reception unit 14 forms reception beams by applying phase addition processing to the plurality of reception signals. The data of the reception beams is output to an image generation unit 16. That is, the transmission/reception unit 14 forms reception beams by performing delay processing on the reception signals obtained from each ultrasound oscillator according to a delay processing condition for each ultrasound oscillator and performing addition processing on the plurality of reception signals obtained from the plurality of ultrasound oscillators. The delay processing condition is defined by reception delay data indicating a delay time. A reception delay data set (that is, a set of delay times) corresponding to the plurality of ultrasound oscillators is supplied from a controller 32.

The electron scanning with the ultrasound beams (that is, the transmission beam and the reception beam) is performed by the action of the transmission/reception unit 14. Thereby, a scanning cross section is formed. The scanning cross section corresponds to a plurality of beams, and a reception frame (specifically, an RF signal frame) is configured with the plurality of beams. Note that each beam is configured with a plurality of echoes arranged in a depth direction. By repeating the electron scanning with the ultrasound beams, a plurality of reception frames arranged on a time axis are output from the transmission/reception unit 14 to the image generation unit 16. A reception frame sequence is configured with the plurality of reception frames.

In a case where the electron scanning with the ultrasound beams is two-dimensionally performed by the action of the transmission/reception unit 14, a three-dimensional echo data acquisition space is formed, and volume data as an echo data set is acquired from the three-dimensional echo data acquisition space. By repeating the electron scanning with the ultrasound beams, a plurality of pieces of volume data that are arranged on a time axis are output from the transmission/reception unit 14 to the image generation unit 16. A volume data sequence is configured with the plurality of pieces of volume data.

The image generation unit 16 generates ultrasound image data (for example, B-mode image data) by applying signal processing, such as wave detection, amplitude compression (for example, logarithmic compression), a coordinate conversion function by a convert function (a digital scan converter (DSC)), an interpolation processing function, and the like, to the reception frame output from the transmission/reception unit 14.

In the following, the image data is appropriately referred to as an “image”. For example, the ultrasound image data is appropriately referred to as an “ultrasound image”, and the B-mode image data is appropriately referred to as a “B-mode image”. Note that the ultrasound image according to the present embodiment is not limited to the B-mode image and may be any image generated by transmission and reception of the ultrasound waves. For example, the ultrasound image according to the present embodiment may be a color doppler image, a pulse doppler image, a strain image, a shear wave elastography image, or the like.

Note that the transmission/reception unit 14 and the image generation unit 16 correspond to an example of an acquisition unit.

A display processing unit 18 generates a display image by overlaying necessary graphic data on the ultrasound image. The display image is output to a display unit 20. One or a plurality of images are arranged and displayed in a display aspect according to a display mode.

The display unit 20 is a display such as a liquid crystal display or an EL display. The ultrasound image such as the B-mode image is displayed on the display unit 20. The display unit 20 may be a device comprising both a display and an operation unit 22. For example, a graphic user interface (GUI) may be realized by the display unit 20 and the operation unit 22. In addition, a user interface such as a touch panel may be realized by the display unit 20 and the operation unit 22.

The operation unit 22 is a device that allows the user to input imaging conditions, commands, and the like to the ultrasound diagnostic apparatus 10. For example, the operation unit 22 is an operation panel, a switch, a button, a keyboard, a mouse, a trackball, or a joystick.

A storage unit 24 includes one or a plurality of storage regions for storing data. For example, the storage unit 24 includes a hard disk drive (HDD), a solid state drive (SSD), various memories (for example, RAM, DRAM, ROM, and the like), other storage devices (for example, an optical disk and the like), or a combination thereof.

For example, the reception signal received by the transmission/reception unit 14, the reception beam generated based on the reception signal, the reception frame, the volume data, the ultrasound image, information indicating the imaging condition, information related to the subject (for example, the patient), and the like are stored in the storage unit 24.

An analysis unit 26 includes a recognition unit 28 and an image quality adjustment unit 30, and determines an imaging condition for adjusting an image quality of the ultrasound image by analyzing the ultrasound image. The analysis unit 26 outputs the information indicating the imaging condition to the transmission/reception unit 14, the image generation unit 16, or both of the transmission/reception unit 14 and the image generation unit 16.

The imaging condition includes at least one condition of a condition related to the transmission/reception unit 14 or a condition related to the image generation unit 16. The condition related to the transmission/reception unit 14 includes a condition related to transmission of the ultrasound waves and a condition related to reception of the ultrasound waves. The condition related to the image generation unit 16 includes a condition related to image processing for generating an ultrasound image. In a case where the imaging condition is the condition related to the transmission/reception unit 14, the analysis unit 26 outputs information indicating the imaging condition to the transmission/reception unit 14. In a case where the imaging condition is the condition related to the image generation unit 16, the analysis unit 26 outputs information indicating the imaging condition to the image generation unit 16. In a case where the imaging condition includes the condition related to the transmission/reception unit 14 and the condition related to the image generation unit 16, the analysis unit 26 outputs information indicating the imaging condition to both the transmission/reception unit 14 and the image generation unit 16.

Specific examples of the imaging condition include parameters such as brightness, contrast, smoothing, an adaptive filter, a gamma value, sharpness, edge enhancement, a gain, a frame rate, a focal depth of a transmission beam, an aperture width, a center frequency of ultrasound waves to be transmitted, a frequency band of ultrasound waves to be transmitted, frequency characteristics of ultrasound waves to be received, a coefficient of an image processing filter (for example, a smoothing filter or the like), a shape of apodization, characteristics of a reception bandpass filter, and characteristics of a dynamic range. Of course, these parameters are merely an example of the imaging condition, and parameters other than these parameters may be included in the imaging condition according to the present embodiment. For example, the imaging condition includes a combination of a plurality of types of parameters.

In a case where the information indicating the imaging condition determined by the analysis unit 26 is output from the analysis unit 26 to the transmission/reception unit 14, the transmission/reception unit 14 controls transmission and reception of the ultrasound waves by the ultrasound probe 12 according to the imaging condition determined by the analysis unit 26.

In a case where the information indicating the imaging condition determined by the analysis unit 26 is output from the analysis unit 26 to the image generation unit 16, the image generation unit 16 adjusts an image quality of the ultrasound image according to the imaging condition determined by the analysis unit 26.

In a case where the information indicating the imaging condition determined by the analysis unit 26 is output to the transmission/reception unit 14 and the image generation unit 16, the transmission/reception unit 14 controls transmission and reception of the ultrasound waves by the ultrasound probe 12 according to the imaging condition determined by the analysis unit 26, and the image generation unit 16 adjusts an image quality of the ultrasound image according to the imaging condition determined by the analysis unit 26.

The recognition unit 28 estimates one or a plurality of candidates of a scanning cross section that is scanned by the ultrasound waves by executing processing (hereinafter, referred to as “cross section recognition processing”) of recognizing a cross section on the ultrasound image. In addition, the recognition unit 28 calculates a reliability degree of the recognition for each of the candidates of the scanning cross section. The reliability degree is a score (that is, certainty or likelihood of the estimation) representing certainty of the estimation.

For example, the recognition unit 28 may estimate a candidate of a scanning cross section that is currently being scanned by executing cross section recognition processing on the ultrasound image that is currently acquired. That is, the recognition unit 28 may estimate a candidate of the scanning cross section in real time.

As another example, the recognition unit 28 may estimate a candidate of the scanning cross section by executing cross section recognition processing on the ultrasound image that is already acquired and is stored in the storage unit 24 of the ultrasound diagnostic apparatus 10 or an external device.

By estimating the candidate of the scanning cross section, it is possible to estimate a candidate of a portion represented in the ultrasound image that has the scanning cross section. That is, the recognition unit 28 can estimate a candidate of a portion that is being scanned with the ultrasound waves by executing cross section recognition processing on the ultrasound image.

As the cross section recognition processing according to the present embodiment, a well-known cross section recognition processing is used. For example, machine learning or artificial intelligence (AI) may be used for the cross section recognition processing. A type of machine learning or artificial intelligence to be used is not limited, and any algorithm or model may be used. For example, a convolutional neural network (CNN), a recurrent neural network (RNN), generative adversarial networks (GAN), linear models, random forests, decision tree learning, a support vector machine (SVM), an ensemble classifier, or other algorithms are used. In addition, an algorithm that does not require learning, such as pattern matching such as template matching, calculation of a correlation coefficient or a similarity, or the like, may be used for the cross section recognition processing.

For example, the recognition unit 28 estimates one or a plurality of candidates of the scanning cross section by executing the cross section recognition processing using machine learning on the ultrasound image, and calculates a reliability degree representing certainty of the estimation using the machine learning for each of the candidates of the scanning cross section. The recognition unit 28 may estimate one or a plurality of candidates of a portion that is being scanned with the ultrasound waves, and may calculate a reliability degree representing certainty of the estimation for each of the candidates of the portion.

The recognition unit 28 may estimate one or a plurality of candidates of the scanning cross section that is being scanned with the ultrasound waves by comparing the ultrasound image (for example, the B-mode image) generated by transmission and reception of the ultrasound waves with a plurality of reference cross section images (for example, the B-mode images), and may calculate a reliability degree representing certainty of the estimation for each of the candidates of the scanning cross section. For example, the candidate of the scanning cross section is estimated by using a technique such as pattern matching.

The reference cross section image is an ultrasound image for estimating a scanning cross section. For example, one or a plurality of reference cross section images for each diagnosis portion are generated in advance, and are stored in the storage unit 24 of the ultrasound diagnostic apparatus 10 or an external device. The reference cross section image representing a certain portion is an ultrasound image generated by scanning a reference scanning cross section intersecting the certain portion with ultrasound waves. For example, a reference scanning cross section is a cross section to be imaged in the ultrasonography, a representative cross section, or the like.

The image quality adjustment unit 30 determines an imaging condition for adjusting an image quality of the ultrasound image. The image quality adjustment unit 30 outputs information indicating the determined imaging condition to the transmission/reception unit 14, the image generation unit 16, or both the transmission/reception unit 14 and the image generation unit 16.

For example, in a case where the recognition unit 28 estimates a candidate of the cross section, the image quality adjustment unit 30 determines an imaging condition suitable for imaging of the estimated candidate of the cross section (that is, an imaging condition suitable for the ultrasonography of the estimated candidate), and outputs information indicating the determined imaging condition.

For example, for each cross section of the diagnosis portion, an imaging condition suitable for imaging of the cross section (that is, an imaging condition suitable for the ultrasonography of the cross section) is predetermined, and information indicating the imaging condition of each cross section is stored in the storage unit 24 or an external device in advance. For example, for each cross section, cross section identification information for identifying the cross section and the information indicating the imaging condition suitable for imaging of the cross section are stored in the storage unit 24 or an external device in advance by being associated with each other in advance. The image quality adjustment unit 30 specifies the imaging condition associated with the candidate of the cross section that is estimated by the recognition unit 28, and outputs information indicating the imaging condition to the transmission/reception unit 14, the image generation unit 16, or both the transmission/reception unit 14 and the image generation unit 16.

For each diagnosis portion, an imaging condition suitable for imaging of the portion (that is, an imaging condition suitable for the ultrasonography of the portion) may be predetermined, and information indicating the imaging condition of each portion may be stored in advance in the storage unit 24 or an external device. For example, for each diagnosis portion, portion identification information for identifying the diagnosis portion and information indicating an imaging condition suitable for imaging of the diagnosis portion are stored in the storage unit 24 or an external device in advance by being associated with each other in advance. The image quality adjustment unit 30 specifies the imaging condition associated with the portion including the candidate of the cross section that is estimated by the recognition unit 28, and outputs information indicating the imaging condition to the transmission/reception unit 14, the image generation unit 16, or both the transmission/reception unit 14 and the image generation unit 16.

The controller 32 controls each unit of the ultrasound diagnostic apparatus 10. In addition, the controller 32 displays various types of information on the display unit 20.

Hereinafter, an operation of the ultrasound diagnostic apparatus 10 will be described in detail.

First, the acquisition unit acquires a first ultrasound image of the subject according to a first imaging condition. That is, the transmission/reception unit 14 transmits and receives the ultrasound waves by the ultrasound probe 12 according to the first imaging condition, and the image generation unit 16 generates the ultrasound image based on the reception frame that is output from the transmission/reception unit 14 according to the first imaging condition. The ultrasound image that is generated according to the first imaging condition is the first ultrasound image.

For example, the first imaging condition is a predetermined imaging condition (for example, a preset condition). The preset condition includes a plurality of types of parameters, and information indicating the preset condition is stored in advance in the storage unit 24 of the ultrasound diagnostic apparatus 10. A plurality of different preset conditions may be created in advance. In this case, a user, such as a doctor or a medical technician, selects the preset condition from among the plurality of different preset conditions by operating the operation unit 22. The selected preset condition is the first imaging condition. The acquisition unit (that is, the transmission/reception unit 14 and the image generation unit 16) acquires the first ultrasound image according to the preset condition that is selected by the user. The user may create the first imaging condition by operating the operation unit 22 and setting the plurality of types of parameters.

In a case where the first ultrasound image is acquired, the recognition unit 28 estimates one or a plurality of candidates of a scanning cross section that is being scanned with the ultrasound waves by executing the cross section recognition processing on the first ultrasound image. In addition, the recognition unit 28 calculates a reliability degree of the recognition for each of the candidates of the scanning cross section.

In a case where a state where the reliability degree of the plurality of candidates having higher reliability degrees satisfies a threshold value condition is continued for a predetermined time T, the acquisition unit acquires a second ultrasound image of the subject according to a second imaging condition. That is, the transmission/reception unit 14 transmits and receives the ultrasound waves by the ultrasound probe 12 according to the second imaging condition, and the image generation unit 16 generates the ultrasound image based on the reception frame that is output from the transmission/reception unit 14 according to the second imaging condition. The ultrasound image acquired according to the second imaging condition is a second ultrasound image.

The second imaging condition is an imaging condition different from the first imaging condition, and is an imaging condition according to the scanning cross section that is recognized by the recognition unit 28. As described above, for each cross section of the diagnosis portion, an imaging condition suitable for imaging of the cross section (that is, an imaging condition suitable for the ultrasonography of the cross section) is predetermined, and information indicating the imaging condition of each cross section is stored in the storage unit 24 or an external device in advance. The image quality adjustment unit 30 specifies the imaging condition associated with the scanning cross section that is recognized by the recognition unit 28 (that is, the candidate estimated by the recognition unit 28), and outputs information indicating the imaging condition to the transmission/reception unit 14, the image generation unit 16, or both the transmission/reception unit 14 and the image generation unit 16. In a case where the imaging condition is set for each diagnostic portion, the image quality adjustment unit 30 specifies the imaging condition associated with the portion including the candidate of the cross section that is estimated by the recognition unit 28, and outputs information indicating the imaging condition to the transmission/reception unit 14, the image generation unit 16, or both the transmission/reception unit 14 and the image generation unit 16.

The time T may be changed by a user such as a doctor or a medical technician. The time T may be determined based on the number of the frames of the ultrasound image. The time T may be determined for each portion as a target of the ultrasonography.

For example, the reliability degree of the plurality of candidates having higher reliability degrees is a sum of the reliability degrees that is calculated by adding the reliability degree of each candidate in order from the candidate having a highest reliability degree. The sum of the reliability degrees is calculated by the image quality adjustment unit 30. The image quality adjustment unit 30 calculates the sum of the reliability degrees by adding the reliability degree of each candidate in order from the candidate having a highest reliability degree until the sum of the reliability degrees exceeds a threshold value. The threshold value is a predetermined value. The threshold value may be changed by the user. The image quality adjustment unit 30 may calculate the sum of the reliability degrees by adding the reliability degrees of a predetermined number of candidates in order from the candidate having a highest reliability degree. The predetermined number may be changed by the user.

The state where the reliability degree of the plurality of candidates having higher reliability degrees satisfies the threshold value condition is a state where the sum of the reliability degrees is within a threshold value range. The threshold value range may be predetermined, or may be changed by the user. The threshold value range is a range between an upper limit threshold value A of the reliability degree and a lower limit threshold value B of the reliability degree. For example, in a case where a state where the sum of the reliability degrees is within the threshold value range is continued for a time T, the acquisition unit acquires the second ultrasound image according to the second imaging condition.

As another example, the state where the reliability degree of the plurality of candidates having higher reliability degrees satisfies the threshold value condition is a state where a moving average value of the sum of the reliability degrees is within a threshold value range. The threshold value range may be predetermined, or may be changed by the user. The threshold value range is a range between an upper limit threshold value C of the moving average value and a lower limit threshold value D of the moving average value. For example, in a case where a state where the moving average value of the sum of the reliability degrees is within the threshold value range is continued for a time T, the acquisition unit acquires the second ultrasound image according to the second imaging condition. The moving average value of the sum of the reliability degrees is calculated by the image quality adjustment unit 30.

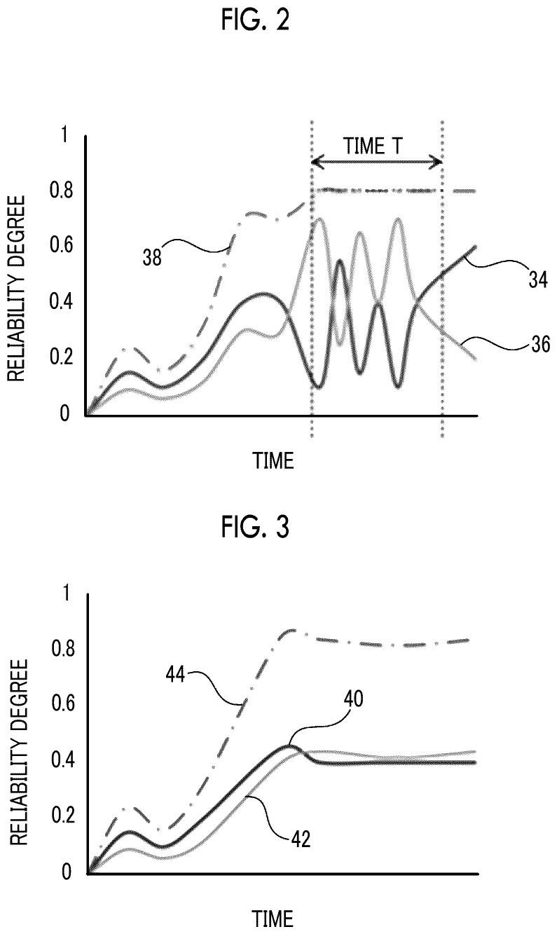

Hereinafter, a temporal change of the reliability degree of the cross section recognition will be described with reference to FIG. 2. FIG. 2 shows a temporal change of the reliability degree of the cross section recognition. A horizontal axis indicates time, and a vertical axis indicates a reliability degree of the cross section recognition.

A reference numeral 34 indicates a temporal change of a reliability degree of a cross section α. A reference numeral 36 indicates a temporal change of a reliability degree of a cross section β. A reference numeral 38 indicates a temporal change in a sum of the reliability degree of the cross section α and the reliability degree of the cross section β.

The cross sections α and β are candidates having higher reliability degrees. For example, the cross section α is a candidate having a highest reliability degree or a second highest reliability degree, and the cross section β is a candidate having a highest reliability degree or a second highest reliability degree. In a case where the reliability degree of the cross section α is highest, the reliability degree of the cross section β is second highest, and in a case where the reliability degree of the cross section α is second highest, the reliability degree of the cross section β is highest. Therefore, the cross sections α and β correspond to candidates having higher reliability degrees. As another example, in a case where the user designates two candidates as candidates having higher reliability degrees, the cross sections α and β are determined as the candidates having higher reliability degrees.

In the example shown in FIG. 2, as indicated by the reference numerals 34 and 36, the reliability degree of the cross section α and the reliability degree of the cross section β change up and down as a time is elapsed, and the rank of the cross section α and the rank of the cross section β are switched as a time is elapsed. For example, in a case where the cross section actually scanned with the ultrasound waves is a cross section having image characteristics of the cross section α and image characteristics of the cross section β, a temporal change of the reliability degree that is indicated by each of the reference numerals 34 and 36 is observed.

FIG. 3 shows another temporal change of the reliability degree of the cross section recognition. A horizontal axis indicates time, and a vertical axis indicates a reliability degree of the cross section recognition.

A reference numeral 40 indicates a temporal change of a reliability degree of a cross section α. A reference numeral 42 indicates a temporal change of a reliability degree of a cross section β. A reference numeral 44 indicates a temporal change in a sum of the reliability degree of the cross section α and the reliability degree of the cross section β.

As in the example shown in FIG. 2, the cross sections α and β are candidates having higher reliability degrees. In a case where the reliability degree of the cross section α is highest, the reliability degree of the cross section β is second highest, and in a case where the reliability degree of the cross section α is second highest, the reliability degree of the cross section β is highest.

In the example shown in FIG. 3, as indicated by the reference numerals 40 and 42, the reliability degree of the cross section α and the reliability degree of the cross section β are equal to or lower than 0.5 (that is, 50%), and do not significantly change up or down as a time is elapsed. For example, in a case where the cross section actually scanned with the ultrasound waves does not have many image characteristics of either of the cross sections α and β, the temporal change indicated by each of the reference numerals 40 and 42 is observed.

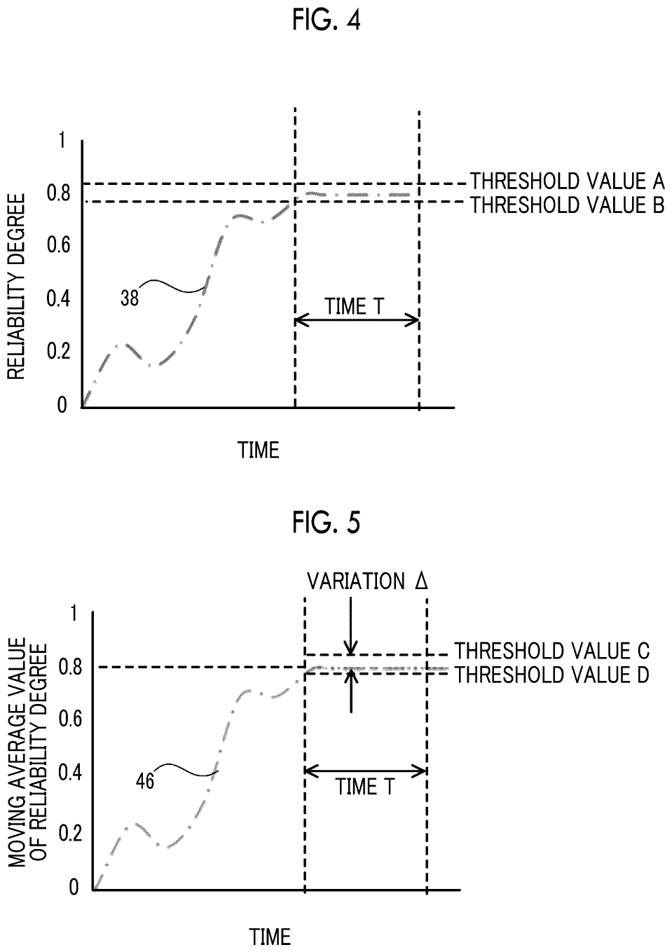

A relationship between the sum of the reliability degrees and the threshold value range will be described with reference to FIG. 4. FIG. 4 shows a temporal change of the sum of the reliability degrees. A horizontal axis indicates time, and a vertical axis indicates a reliability degree of the cross section recognition. In addition, a time T, an upper limit threshold value A, and a lower limit threshold value B are shown in FIG. 4.

A reference numeral 38 indicates a temporal change in a sum of the reliability degree of the cross section α and the reliability degree of the cross section β. The graph indicated by the reference numeral 38 in FIG. 4 is the same as the graph indicated by the reference numeral 38 in FIG. 2.

As shown in FIG. 2 and FIG. 4, the sum of the reliability degrees is greatly changed in a time zone before the time T. However, thereafter, the sum of the reliability degrees fluctuates between the upper limit threshold value A and the lower limit threshold value B for the time T. In the examples shown in FIG. 2 and the FIG. 4, the sum of the reliability degrees fluctuates between the upper limit threshold value A and the lower limit threshold value B for the time T. In a case where the state where the sum of the reliability degrees is within the threshold value range (that is, between the upper limit threshold value A and the lower limit threshold value B) is continued for the time T, the acquisition unit acquires the second ultrasound image according to the second imaging condition.

The sum of the reliability degrees that is indicated by the reference numeral 44 in FIG. 3 is also greatly changed in a time zone before the time T. However, thereafter, the sum of the reliability degrees fluctuates between the upper limit threshold value A and the lower limit threshold value B for the time T. Therefore, the acquisition unit acquires the second ultrasound image according to the second imaging condition.

A relationship between the moving average value of the sum of the reliability degrees and the threshold value range will be described with reference to FIG. 5. FIG. 5 shows a temporal change of the moving average value of the sum of the reliability degrees. A horizontal axis indicates a time, and a vertical axis indicates a moving average value of the reliability degree of the cross section recognition. Further, the time T, the upper limit threshold value C, the lower limit threshold value D, and a variation A are shown in FIG. 5.

For example, the image quality adjustment unit 30 calculates a moving average value of the sum of the reliability degrees indicated by the reference numeral 38 in FIG. 2. A reference numeral 46 indicates a temporal change of the moving average value of the sum of the reliability degrees indicated by the reference numeral 38. For example, the variation A is a variation of the moving average value, and is approximately 10% of the moving average value. Of course, this value is merely an example. Another value may be used as the variation A, or the variation A may be set by the user.

As shown in FIG. 5, the moving average value of the sum of the reliability degrees is greatly changed in a time zone before the time T. However, thereafter, the moving average value of the sum of the reliability degrees fluctuates between the upper limit threshold value C and the lower limit threshold value D for the time T. In addition, a variation in the moving average value is within the variation A. In a case where the state where the moving average value of the sum of the reliability degrees is within the threshold value range (that is, between the upper limit threshold value C and the lower limit threshold value D) is continued for the time T, the acquisition unit acquires the second ultrasound image according to the second imaging condition.

In general, in a case where a user such as a doctor or a medical technician searches for a cross section suitable for the ultrasonography while performing imaging using the ultrasound probe 12, it is considered that the reliability degree of the estimation of the cross section is not stable. For example, during the search of the cross section, the user may perform imaging while changing a position or an angle of the ultrasound probe 12. In this case, it is considered that a variation in the reliability degree calculated during the search is large and the reliability degree is not stable. On the other hand, in a case where the user continues to perform imaging without changing the position or the angle of the ultrasound probe 12, it is considered that a change in the reliability degree that is calculated during the imaging is small and the reliability degree is stable.

For example, in a case where a state where the sum of the reliability degrees is within the threshold value range is continued for a time T, it is presumed that the sum of the reliability degrees is stable. Similarly, in a case where a state where the moving average value of the sum of the reliability degrees is within the threshold value range is continued for the time T, it is presumed that the sum of the reliability degrees is stable. That is, in these cases, it is presumed that a certain cross section is stably scanned. Therefore, the acquisition unit acquires the second ultrasound image according to the second imaging condition. As a result, it is possible to improve the reliability degree of the recognition of the cross section.

The case where the state where the reliability degree of the plurality of candidates having higher reliability degrees satisfies the threshold value condition is continued for a time T may be a case where a state where the ranks of the plurality of candidates having higher reliability degrees do not change is continued for a time T and the reliability degree of each of the plurality of candidates having higher reliability degrees is lower than the threshold value. The threshold value is a predetermined value. The threshold value may be changed by the user. For example, it is presumed that, in a case where a state where the reliability degree of the cross section α is highest and the reliability degree of the cross section β is second highest is continued for a time T and each of the reliability degree of the cross section α and the reliability degree of the cross section β is lower than the threshold value, a certain cross section is stably scanned. Therefore, the acquisition unit acquires the second ultrasound image according to the second imaging condition.

Hereinafter, a specific example of the second imaging condition will be described.

For example, the second imaging condition is an imaging condition corresponding to a candidate of which the reliability degree exceeds the threshold value among the plurality of candidates having higher reliability degrees. The threshold value is a predetermined value. The threshold value may be changed by the user.

In the examples shown in FIG. 2 and FIG. 3, in a case where the reliability degree of the cross section α exceeds the threshold value and the reliability degree of the cross section β does not exceed the threshold value, the image quality adjustment unit 30 specifies, as the second imaging condition, an imaging condition (that is, an imaging condition associated with the cross section α) suitable for imaging of the cross section α, and outputs information indicating the imaging condition to the transmission/reception unit 14, the image generation unit 16, or both the transmission/reception unit 14 and the image generation unit 16. Thereby, the acquisition unit acquires the second ultrasound image according to the second imaging condition corresponding to the cross section α.

In a case where the reliability degree of the cross section α does not exceed the threshold value and the reliability degree of the cross section β exceeds the threshold value, the image quality adjustment unit 30 specifies, as the second imaging condition, an imaging condition (that is, an imaging condition associated with the cross section β) suitable for imaging of the cross section β, and outputs information indicating the imaging condition to the transmission/reception unit 14, the image generation unit 16, or both the transmission/reception unit 14 and the image generation unit 16. Thereby, the acquisition unit acquires the second ultrasound image according to the second imaging condition corresponding to the cross section β.

The second imaging condition may be an imaging condition corresponding to a candidate having a highest reliability degree among the plurality of candidates having higher reliability degrees. For example, in a case where both the reliability degree of the cross section α and the reliability degree of the cross section β exceed the threshold value, the image quality adjustment unit 30 specifies, as the second imaging condition, an imaging condition corresponding to the cross section having the highest reliability degree between the cross section α and the cross section β.

In a case where the reliability degree of the cross section α is higher than the reliability degree of the cross section β, the image quality adjustment unit 30 specifies, as the second imaging condition, an imaging condition (that is, an imaging condition associated with the cross section α) suitable for imaging of the cross section α, and outputs information indicating the imaging condition to the transmission/reception unit 14, the image generation unit 16, or both the transmission/reception unit 14 and the image generation unit 16. Thereby, the acquisition unit acquires the second ultrasound image according to the second imaging condition corresponding to the cross section α.

In a case where the reliability degree of the cross section β is higher than the reliability degree of the cross section α, the image quality adjustment unit 30 specifies, as the second imaging condition, an imaging condition (that is, an imaging condition associated with the cross section β) suitable for imaging of the cross section β, and outputs information indicating the imaging condition to the transmission/reception unit 14, the image generation unit 16, or both the transmission/reception unit 14 and the image generation unit 16. Thereby, the acquisition unit acquires the second ultrasound image according to the second imaging condition corresponding to the cross section β.

According to the present embodiment, the user such as a doctor or a medical technician can perform ultrasonography using the second ultrasound image suitable for the ultrasonography. As a result, the user does not need to set the imaging condition suitable for the diagnosis portion, and it is possible to reduce a burden and a time for setting the imaging condition.

Hereinafter, application examples of the present embodiment will be described.

For example, in ultrasonography for a liver, in a case where the imaging condition is not appropriate, a portal vein may be difficult to be visualized on an ultrasound image showing a cross section between ribs of the liver due to insufficient sensitivity or the like. As a result, the reliability degree of the cross section recognition may be decreased. According to the present embodiment, for example, in a case where a state where the sum of the reliability degrees of two cross sections having highest reliability degrees is within the threshold value range is continued for a time T, the acquisition unit acquires the second ultrasound image according to the second imaging condition. For example, an imaging condition suitable for imaging of a cross section between ribs of a liver (for example, an imaging condition in which a focal depth, an aperture width, a gain, and the like of the transmission beam are adjusted) is used as the second imaging condition, and the second ultrasound image is acquired. Thereby, high sensitivity is obtained, and the second ultrasound image in which the blood vessel is appropriately visualized is generated. In addition, the second ultrasound image having a high reliability degree is generated.

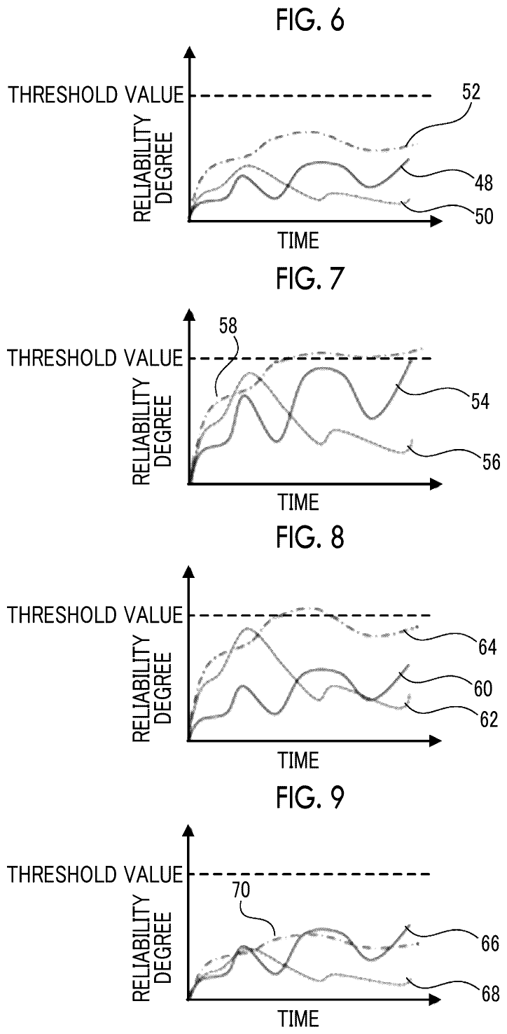

Hereinafter, specific examples of the second imaging condition will be described with reference to FIG. 6 to FIG. 9. FIG. 6 to FIG. 9 show a temporal change of the reliability degree of the cross section recognition. In FIG. 6 to FIG. 9, a horizontal axis indicates a time, and a vertical axis indicates a reliability degree.

FIG. 6 shows a temporal change of the reliability degree calculated from the first ultrasound image. A reference numeral 48 indicates a temporal change of the reliability degree of the cross section α. A reference numeral 50 indicates a temporal change of the reliability degree of the cross section β. A reference numeral 52 indicates a temporal change in the sum of the reliability degree of the cross section α and the reliability degree of the cross section β. Each reliability degree is a value calculated from the first ultrasound image. In a case where the state where the sum of the reliability degrees satisfies the threshold value condition is continued for a time T, the acquisition unit acquires the second ultrasound image according to the second imaging condition.

FIG. 7 to FIG. 9 show a temporal change of the reliability degree calculated from the second ultrasound image.

The reliability degree of each cross section shown in FIG. 7 is a reliability degree calculated from the second ultrasound image acquired according to the second imaging condition in which the contrast is given priority. A reference numeral 54 indicates a temporal change of the reliability degree of the cross section α. A reference numeral 56 indicates a temporal change of the reliability degree of the cross section β. A reference numeral 58 indicates a temporal change in the sum of the reliability degree of the cross section α and the reliability degree of the cross section β.

The reliability degree of each cross section shown in FIG. 8 is a reliability degree calculated from the second ultrasound image acquired according to the second imaging condition in which the resolution is given priority. A reference numeral 60 indicates a temporal change of the reliability degree of the cross section α. A reference numeral 62 indicates a temporal change of the reliability degree of the cross section β. A reference numeral 64 indicates a temporal change in the sum of the reliability degree of the cross section α and the reliability degree of the cross section β.

The reliability degree of each cross section shown in FIG. 9 is a reliability degree calculated from the second ultrasound image acquired according to the second imaging condition in which sensitivity in a deep portion is given priority. A reference numeral 66 indicates a temporal change of the reliability degree of the cross section α. A reference numeral 68 indicates a temporal change of the reliability degree of the cross section β. A reference numeral 70 indicates a temporal change in the sum of the reliability degree of the cross section α and the reliability degree of the cross section β.

Hereinafter, modification examples will be described.

### Modification Example 1

In the modification example 1, the recognition unit 28 estimates one or a plurality of candidates of a scanning cross section by executing the cross section recognition processing on the second ultrasound image. In addition, the recognition unit 28 calculates a reliability degree of the recognition for each of the candidates of the scanning cross section.

The image quality adjustment unit 30 compares the reliability degree calculated from the first ultrasound image with the reliability degree calculated from the second ultrasound image. For example, the image quality adjustment unit 30 compares the highest reliability degree that is the reliability degree calculated from the first ultrasound image (that is, the reliability degree of the cross section having the highest reliability degree) with the highest reliability degree that is the reliability degree calculated from the second ultrasound image (that is, the reliability degree of the cross section having the highest reliability degree).

In a case where the reliability degree calculated from the second ultrasound image is higher than the reliability degree calculated from the first ultrasound image, the image quality adjustment unit 30 outputs the information indicating the second imaging condition to the transmission/reception unit 14, the image generation unit 16, or both the transmission/reception unit 14 and the image generation unit 16. The acquisition unit acquires the second ultrasound image according to the second imaging condition.

In a case where the reliability degree calculated from the second ultrasound image is equal to or lower than the reliability degree calculated from the first ultrasound image, the image quality adjustment unit 30 maintains the first imaging condition as the imaging condition without changing the imaging condition. The acquisition unit continues ultrasonography imaging according to the first imaging condition.

According to the modification example 1, the ultrasound image is acquired according to the imaging condition under which a higher reliability degree is obtained, and thus, it is possible to realize more appropriate ultrasonography.

### Modification Example 2

In the modification example 2, the controller 32 displays the first ultrasound image and the second ultrasound image on the display unit 20. For example, the controller 32 displays the first ultrasound image and the second ultrasound image on the display unit 20 side by side. The controller 32 may switch the first ultrasound image and the second ultrasound image, and may display the first ultrasound image and the second ultrasound image on the display unit 20. For example, in a case where the user inputs a switching instruction by operating the operation unit 22, the controller 32 switches the first ultrasound image and the second ultrasound image, and displays the first ultrasound image and the second ultrasound image on the display unit 20. The controller 32 may automatically switch the first ultrasound image and the second ultrasound image each time a predetermined time elapses, and may display the first ultrasound image and the second ultrasound image on the display unit 20.

The user selects the first imaging condition or the second imaging condition by operating the operation unit 22 with reference to the first ultrasound image and the second ultrasound image displayed on the display unit 20. The acquisition unit acquires the ultrasound image according to the imaging condition selected by the user.

For example, the controller 32 displays an image (for example, an icon or a button image representing the first imaging condition) for selecting the first imaging condition and an image (for example, an icon or a button image representing the second imaging condition) for selecting the second imaging condition side by side on the display unit 20. The user selects the imaging condition by pressing an icon, a button image, or the like.

As another example, the user may select the imaging condition by selecting the displayed ultrasound image, and the image quality adjustment unit 30 may receive the selection. For example, in a case where the user selects the first ultrasound image by operating the operation unit 22, the image quality adjustment unit 30 receives the selection and sets the imaging condition to the first imaging condition. In a case where the user selects the second ultrasound image by operating the operation unit 22, the image quality adjustment unit 30 receives the selection and sets the imaging condition to the second imaging condition. The acquisition unit acquires the ultrasound image according to the imaging condition that is set by the image quality adjustment unit 30.

According to the modification example 2, the ultrasound image is acquired according to the imaging condition that is intended by the user. Therefore, the ultrasound image intended by the user is generated and is presented to the user.

In a case where the reliability degree calculated from the second ultrasound image is lower than the reliability degree calculated from the first ultrasound image, the controller 32 may not display the second ultrasound image on the display unit 20. In this case, in a case where the reliability degree calculated from the second ultrasound image is higher than the reliability degree calculated from the first ultrasound image, the controller 32 displays the first ultrasound image and the second ultrasound image on the display unit 20.

### Modification Example 3

In the modification example 3, the second imaging condition is a condition related to the image processing by the image generation unit 16. The image quality adjustment unit 30 outputs information indicating the second imaging condition to the image generation unit 16. The image generation unit 16 generates the second ultrasound image by executing image processing according to the second imaging condition on the data (for example, the reception frame) acquired according to the first imaging condition. The image processing according to the second imaging condition is image processing suitable for the estimated cross section, and is, for example, image processing in which an image processing filter (for example, a smoothing filter) is adjusted.

According to the modification example 3, the second ultrasound image can be acquired by changing the condition of the image processing using the data acquired according to the first imaging condition.

The image generation unit 16, the display processing unit 18, the analysis unit 26, and the controller 32 can be realized by using, for example, hardware resources such as a processor and an electronic circuit, and a device such as a memory may be used as necessary in the realization. In addition, the image generation unit 16, the display processing unit 18, the analysis unit 26, and the controller 32 may be realized by, for example, a computer. That is, all or a part of the image generation unit 16, the display processing unit 18, the analysis unit 26, and the controller 32 may be realized by cooperation between hardware resources, such as a central processing unit (CPU) or a memory included in a computer, and software (program) that defines the operation of the CPU or the like. The program is stored in the storage unit 24 of the ultrasound diagnostic apparatus 10 or another storage device through a recording medium such as a CD or a DVD, or through a communication path such as a network. As another example, the image generation unit 16, the display processing unit 18, the analysis unit 26, and the controller 32 may be realized by a digital signal processor (DSP), an application specific integrated circuit (ASIC), a field programmable gate array (FPGA), or the like. Of course, a graphics processing unit (GPU) or the like may be used. The image generation unit 16, the display processing unit 18, the analysis unit 26, and the controller 32 may be realized by a single device, or may be realized by a plurality of devices.

The functions of each of the image generation unit 16, the display processing unit 18, the analysis unit 26, and the controller 32 may be executed by a device (for example, a personal computer or a server) other than the ultrasound diagnostic apparatus 10.

What is claimed is:

1. An ultrasound diagnostic apparatus comprising:
an acquisition unit that acquires a first ultrasound image of a subject according to a first imaging condition; and
a recognition unit that executes processing of recognizing a cross section on the first ultrasound image and outputs a plurality of candidates for the cross section and a reliability degree of the recognition for each of the candidates,
wherein, in a case where a state where the reliability degree of the plurality of candidates having higher reliability degrees satisfies a threshold value condition is continued for a predetermined time, the acquisition unit acquires a second ultrasound image of the subject according to a second imaging condition according to the recognized cross section, the second imaging condition being different from the first imaging condition.

2. The ultrasound diagnostic apparatus according to claim 1,
wherein the reliability degree of the plurality of candidates having higher reliability degrees is a sum of the reliability degrees calculated by adding the reliability degree of each candidate in an order from a candidate having a highest reliability degree, and
the case where the state where the reliability degree of the plurality of candidates having higher reliability degrees satisfies the threshold value condition is continued for the predetermined time is a case where a state where the sum is within a threshold value range is continued for a predetermined time.

3. The ultrasound diagnostic apparatus according to claim 1,
wherein the case where the state where the reliability degree of the plurality of candidates having higher reliability degrees satisfies the threshold value condition is continued for the predetermined time is a case where a state where ranks of the plurality of candidates having higher reliability degrees are not changed is continued for a predetermined time and the reliability degree of each of the plurality of candidates having higher reliability degrees is lower than a threshold value.

4. The ultrasound diagnostic apparatus according to claim 1,
wherein the second imaging condition is an imaging condition corresponding to a candidate of which the reliability degree is higher than a threshold value among the plurality of candidates having higher reliability degrees.

5. The ultrasound diagnostic apparatus according to claim 4,
wherein the second imaging condition is an imaging condition corresponding to a candidate having a highest reliability degree among the plurality of candidates having higher reliability degrees.

6. The ultrasound diagnostic apparatus according to claim 1,
wherein the recognition unit further executes processing of recognizing a cross section on the second ultrasound image, and calculates a reliability degree of the recognition, and
the acquisition unit further acquires the second ultrasound image according to the second imaging condition in a case where the reliability degree calculated from the second ultrasound image is higher than the reliability degree calculated from the first ultrasound image.

7. The ultrasound diagnostic apparatus according to claim 6,
wherein the acquisition unit further continues ultrasonography according to the first imaging condition in a case where the reliability degree calculated from the second ultrasound image is equal to or lower than the reliability degree calculated from the first ultrasound image.

8. The ultrasound diagnostic apparatus according to claim 1, further comprising:
a controller that displays the first ultrasound image and the second ultrasound image on a display,
wherein the acquisition unit acquires an ultrasound image according to an imaging condition selected by a user from the first imaging condition and the second imaging condition.

9. The ultrasound diagnostic apparatus according to claim 1,
wherein the second imaging condition is a condition related to image processing, and
the acquisition unit acquires the second ultrasound image by executing image processing according to the second imaging condition, on data acquired according to the first imaging condition.

10. A non-transitory computer-readable storage medium storing a program causing a computer to function as:
an acquisition unit that acquires a first ultrasound image of a subject according to a first imaging condition; and
a recognition unit that executes processing of recognizing a cross section on the first ultrasound image and outputs a plurality of candidates for the cross section and a reliability degree of the recognition for each of the candidates,
wherein, in a case where a state where the reliability degree of the plurality of candidates having higher reliability degrees satisfies a threshold value condition is continued for a predetermined time, the acquisition unit acquires a second ultrasound image of the subject according to a second imaging condition according to the recognized cross section, the second imaging condition being different from the first imaging condition.
